(19) United States
(12) Patent Application Publication
(10) Pub. No.: US 2026/0174369 A1
(43) Pub. Date: Jun. 25, 2026

(54) SYSTEMS AND METHODS TO RECORD BIOMAGNETIC SIGNALS AT AMBIENT CONDITIONS

(71) Applicant: Sonera, Inc., Berkeley, CA (US)

(72) Inventors: Richy YUN, Richmond, CA (US); Debadatta DASH, Berkeley, CA (US); Isabel GERRARD, Oakland, CA (US); Dominic LABANOWSKI, Berkeley, CA (US)

(21) Appl. No.: 19/329,554

(22) Filed: Sep. 15, 2025

Related U.S. Application Data
(60) Provisional application No. 63/721,401, filed on Nov. 15, 2024.

(51) Int. Cl. A61B 5/242; A61B 5/00

(52) U.S. Cl. CPC A61B 5/242; A61B 5/7203; A61B 2562/046; A61B 2562/18; A61B 2562/227

(57) ABSTRACT

Described herein are methods and apparatuses to provide accurate measurement of relatively small magnetic fields under ambient conditions while being worn on an ambulatory subject. These methods and apparatuses may allow accurate and reliable detection of biomagnetic fields from organs such as the brain, heart, and muscles, using a magnetometer, and in particular, using an Acoustically Driven Ferromagnetic Resonance (ADFMR) sensor. These methods and apparatuses may incorporate one or more of: a synthetic gradiometer, flexible and/or thin-film/foil shielding, and/or denoising.

### CLAIM OF PRIORITY

This patent application claims priority to U.S. Provisional Ser. No. 63/721,401, titled “SYSTEMS AND METHODS TO RECORD BIOMAGNETIC SIGNALS AT AMBIENT CONDITIONS,” filed on Nov. 15, 2024, which is herein incorporated by reference in its entirety.

### BACKGROUND

Sensing of biomagnetism, e.g., magnetic fields generated by the body, allows for specific noninvasive recordings of various organs such as the brain, heart, and muscles. Although biomagnetism has been demonstrated to contain richer and more salient information compared to noninvasive electrical recordings, there has yet to be a functional application in unshielded, ambient conditions due to two major limitations. First, the earth's magnetic field as well as other noise sources typically result in saturation of the sensor or are too large making it difficult to extract the signal of interest from noise. Second, available sensors are limited in either sensitivity or size preventing high density layouts that would unlock signal processing techniques to overcome ambient noise.

It would be very useful to provide methods and apparatuses for accurately and reliably measuring biomagnetic signals from a body, e.g., a human or animal body. Described herein are methods and apparatuses that may resolve these problems and may allow accurate measurement of such biomagnetic signals.

### SUMMARY OF THE DISCLOSURE

The methods and apparatuses described herein implement both hardware and software techniques to measure biomagnetic signals by adapting Acoustically Driven Ferromagnetic Resonance (ADFMR) magnetometers. The ADFMR magnetometers may be small in size (˜5×5 mm), enabling high density arrays allowing for optimized synthetic gradiometry as well as improved feature extraction and spatial filtering. These apparatuses (e.g., devices, system, etc. including in particular ADFMR magnetometers) may include small, light, and mobile shielding to attenuate environmental noise. The methods and apparatuses described herein may also use a unique signal processing pipeline to isolate biosignals and maximize signal to noise ratio. Using these methods and apparatuses, ambient recordings of heart signals (magnetocardiography (MCG)) have been demonstrated using 14 sensors with much lower sensitivity than previously shown to be feasible. These methods and apparatuses have also been used to record accurate muscle activity (magnetomyography, MMG) in ambient conditions for the first time, enabling their use towards various applications.

For example, described herein are methods of sensing a biomagnetic field under ambient conditions in an ambulatory subject. In some cases, these method include: receiving a primary signal from one or more primary acoustically driven ferromagnetic resonance (ADFMR) sensors worn on the subject; receiving a secondary signal from one or more secondary ADFMR sensor worn on the subject; weighting the secondary signal by a weighting factor; periodically adjusting, at a tuning frequency (e.g., range of frequencies), the weighting factor; generating a corrected biomagnetic sensor signal by subtracting the primary signal from the weighted secondary signal; and outputting the corrected biomagnetic sensor signal.

Any of these method may include estimating a level of noise from a spectral density of the primary and/or secondary signal and weighting the secondary signal to minimize the estimated noise. For example, weighting the secondary signal to minimize the estimated noise may comprise using a gradient-based and/or stochastic technique to minimize the estimate noise.

In some cases receiving the secondary signal comprises receiving the secondary signal from the one or more secondary ADFMR sensor that is flexibly connected to the one or more primary ADFMR sensor.

Any of these methods may include continuously outputting the corrected biomagnetic sensor signal. Any of these methods may include outputting in real time (or near-real time). The one or more secondary ADFMR sensors may comprise three or more secondary ADFMR sensors that are arranged orthogonal to each other. The tuning frequency may be adjusted based on input from a motion sensor. The tuning frequency may be between 0.1 Hz and 10 Hz. Any of these methods may include converting the primary signal and the secondary signal into the digital domain before generating the corrected biomagnetic signal. Any of these methods may include shielding the one or more primary and one or more secondary ADFMR sensors. For example, shielding may comprise shielding under a mumetal foil.

Any of these methods may include denoising the corrected biomagnetic sensor.

For example, a method of sensing a biomagnetic field under ambient conditions in an ambulatory subject may include: receiving a primary signal from one or more primary acoustically driven ferromagnetic resonance (ADFMR) sensor worn on the subject; receiving a secondary signal from one or more secondary ADFMR sensor worn on the subject; estimating a level of noise from a spectral density of the primary and/or secondary signal; weighting the secondary signal by a weighting factor to minimize the estimated noise using a gradient-based and/or stochastic technique; periodically adjusting, at a tuning frequency (e.g., range of frequencies), the weighting factor; generating a corrected biomagnetic sensor signal by subtracting the primary signal from the weighted secondary signal; and outputting the corrected biomagnetic sensor signal.

Also described herein are apparatuses, including apparatuses for sensing a biomagnetic field under ambient conditions in an ambulatory subject. For example, an apparatus may include: one or more primary acoustically driven ferromagnetic resonance (ADFMR) sensor configured to be worn on the subject and to emit a primary signal; one or more secondary ADFMR sensors configured to be worn on the subject and to emit a secondary signal; one or more processors; and a memory coupled to the one or more processors, the memory storing computer-program instructions, that, when executed by the one or more processors, perform a computer-implemented method comprising: weighting the secondary signal by a weighting factor; periodically adjusting, at a tuning frequency (e.g., range of frequencies), the weighting factor; generating a corrected biomagnetic sensor signal by subtracting the primary signal from the weighted secondary signal; and outputting the corrected biomagnetic sensor signal.

These apparatuses may perform any of the steps of the methods described herein, including estimating a level of noise from a spectral density of the primary and/or secondary signal and weighting the secondary signal to minimize the estimated noise. In some cases weighting the secondary signal to minimize the estimated noise comprises using a gradient-based and/or stochastic technique to minimize the estimate noise. The one or more secondary ADFMR sensors may be flexibly connected to the one or more primary ADFMR sensor. The one or more secondary ADFMR sensor and the one or more primary ADFMR sensors may be 5×5 mm or smaller. As mentioned, outputting may be continuously outputting and/or outputting in real time. The one or more secondary ADFMR sensors may comprise three or more secondary ADFMR sensors that are arranged orthogonal to each other.

The tuning frequency may be adjusted based on input from a motion sensor. For example, the tuning frequency may be between 0.1 Hz and 10 Hz.

The apparatus may be further configured to convert the primary signal and the secondary signal into the digital domain before generating the corrected biomagnetic signal.

The apparatus may be configured to denoise the corrected biomagnetic sensor.

Any of these apparatuses may include shielding configured to magnetically shield the primary and one or more secondary ADFMR sensors. For example, the shielding may be a mumetal foil.

For example, an acoustically driven ferromagnetic resonance (ADFMR) sensor system may include: one or more ADFMR sensors; and a flexible foil shield having at least partially covering the ADFMR sensor when the one or more ADFMR sensors is worn on a subject's body. The foil shield may comprise a mumetal foil. The foil shield may be between 0.01 and 0.2 mm thick. In some examples the foil shield is approximately 0.1 mm thick. The foil shield may comprise between 1-4 layers of foil. The foil shield may not completely cover the ADFMR sensor. (e.g., may cover just 20%, 30%, 40%, 50%, 60%, 70%, 80%, 90%, etc.). The foil shield is configured as a flexible wrap configured to be wrapped around the ADFMR sensor and a portion of a person's body to which the sensor is attached.

Also described herein are methods of sensing a biomagnetic field under ambient conditions in an ambulatory subject, the method comprising: placing an acoustically driven ferromagnetic resonance (ADFMR) sensor on a region of a subject's body; shielding, using a flexible foil shield, the ADFMR sensor by at least partially covering the ADFMR sensor with the flexible foil shield; receiving a signal from the shielded ADFMR sensor worn on the subject; generating a biomagnetic sensor signal from the signal; and outputting the biomagnetic sensor signal.

Any of these methods may include shielding comprises partially shielding the ADFMR sensor. In some examples the flexible foil shield comprises a mumetal foil. The flexible foil shield may be between 0.01 and 0.2 mm thick. The flexible foil shield may be approximately 0.1 mm thick or smaller (e.g., 0.09, 0.08, 0.07, 0.05, 0.05 mm thick or thinner). The flexible foil shield may comprise between 1-4 layers of foil. In some cases the flexible foil shield does not completely cover the ADFRM sensor.

A method of sensing a biomagnetic field under ambient conditions in an ambulatory subject may include: placing an acoustically driven ferromagnetic resonance (ADFMR) sensor on a region of a subject's body; receiving a signal from the shielded ADFMR sensor worn on the subject; denoising the signal; generating a biomagnetic sensor signal from the denoised signal; and outputting the biomagnetic sensor signal.

Denoising the signal may comprise time-domain filtering the signal to remove line noise (60 Hz) and/or its harmonics with an IIR notch filter or spectrum interpolation. In some examples denoising the signal comprises applying a two-step noise reduction (TSNR) technique similar to a Wiener filter to remove additive noise. In some examples, denoising the signal comprises using an independent component analysis (ICA), principal component analysis (PCA), and/or another source separation technique to separate signals from noise. For example, denoising the signal may comprise spatial filtering.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a schematical illustration of one example of an ADFMR magnetometer apparatus including a pair of ADFMR sensors configured to use synthetic gradiometry to improve sensing.

FIG. 2 schematically illustrates another example of an apparatus including a plurality of ADFMR sensors configured to use synthetic gradiometry to enhance signal to noise ratio.

FIG. 3A shows frequency-domain comparisons of baseline recordings, gradiometry pairs, and double gradiometry from an example of a magnetometer. The results were normalized to the applied tone at 100 Hz.

FIG. 3B shows the relative reduction in noise with gradiometry from the system tested in FIG. 3A.

FIG. 3C shows an example of SNR of the 100 Hz tone at baseline and with gradiometry from the system tested in FIG. 3A. Two different signals were captured and show higher gain for the smaller signal.

FIG. 4 shows an example of one variation of a single sensor with a flexible (e.g., foil) shielding. In this example, 2 layers of shielding are shown.

FIGS. 5A-5B illustrate graphs showing the noise recorded in ambient conditions with and without mumetal shielding (FIG. 5A) and quantification of the reduction in noise (FIG. 5B). Shielding was performed with 0.1 mm thick mumetal foil.

FIGS. 6A-6C show a comparison of ambient noise in a magnetometer, comparing the signal without denoising (FIG. 6A) under ambient conditions, under the same conditions (ambient) with denoising as described herein (FIG. 6B) and without denoising, but in a shielded room (FIG. 6C).

FIG. 7A shows an example of a set of average heart (e.g., MCG) recordings made in ambient conditions using an apparatus as described herein.

FIG. 7B shows an estimate of heartbeats per minute calculated using magnetic data collected from an apparatus as described herein.

FIG. 7C shows an example of real-time magnetic signals from a patient's heart recorded non-invasively using an apparatus as described herein.

FIGS. 8A-8B show examples of raw (FIG. 8A) MMG traces recording using an apparatus as described herein, and denoised (FIG. 8B) MMG traces taken from a subject's arm before, during and after clenching of the muscle.

### DETAILED DESCRIPTION

The methods and apparatuses (e.g., devices, systems, etc., including hardware, software and/or firmware) described herein are configured to allow for accurate measurement of relatively small magnetic fields under ambient conditions, including under the earth's magnetic field, while being worn on an ambulatory subject. These methods and apparatuses may allow, for the first time, the accurate and reliable detection of biomagnetic fields from organs such as the brain, heart, and muscles, using a magnetometer, and in particular, using an Acoustically Driven Ferromagnetic Resonance (ADFMR) sensor, also referred to herein as an ADFMR magnetometer. Thus, these methods and apparatuses may allow the detection of the complex yet highly relevant biomagnetic information, which may provide both different and potentially more useful information even as compared to other noninvasive sensing techniques, including non-invasive electrical recordings.

As mentioned, these methods and apparatuses may, for the first time, allow accurate and reliable detection of biomagnetic signals in ambient conditions, which was previously not believed possible. The methods and apparatuses described herein may accurately eliminate ambient magnetic fields, including the earth's magnetic field as well as other noise sources that may otherwise saturate magnetic sensors. These methods and apparatuses may be sufficiently small (e.g., 40 mm² or less, 35 mm² or less, 30 mm² or less, 25 mm² or less, 22 mm² or less, 20 mm² or less, etc.) to allow them to be operated in high density layouts that allow signal processing techniques to overcome ambient noise, such as arrays of 3 or more, 4 or more, 5 or more, 6 or more, 7 or more, 8 or more, 9 or more, 10 or more, 14 or more, 25 or more, 30 or more 32 or more, etc. (e.g., 10-20 sensors, 14-16 sensors, 25-35 sensors, etc.).

The apparatuses described herein may include hardware and/or software techniques to allow sensing low magnitude magnetic signals in ambient conditions. As mentioned, these methods and apparatuses may be used with one or more ADFMR sensors. For example, in any of these methods and apparatuses the sensors may be configured as an array of small (e.g., less than 30 mm², such as ˜5×5 mm) ADFMR sensors that are configured to operate as a high density array providing optimized synthetic gradiometry as well as improved feature extraction and spatial filtering. Any of these methods and apparatuses may also or alternatively include ADFMR sensors having shielding specific to the ADFMR sensor, such as including small, light, and/or mobile shielding for each ADFMR sensor configured to attenuate environmental noise (e.g., ambient noise). This shielding may be applied to individual sensors, to groups of sensors within the array, and/or to the full array of sensors. Any of these methods and apparatuses may also include signal processing for the output of the ADFMR sensor(s) to isolate signals (e.g., biosignals) from the background and maximize signal to noise ratio (SNR). Thus the use of these methods and apparatuses may provide highly sensitive and reliable, non-invasive detection of ambient signals. For example, these methods and apparatuses as described herein have been successfully used to take ambient recordings of heart signals with an array of 14 ADFMR sensors (e.g., magnetocardiography, MCG) using much lower sensitivity than previously shown to be feasible. These methods have also been used to record muscle activity (magnetomyography, MMG) in ambient conditions for the first time.

Thus, described herein are high-density arrays of ADFMR sensors. These high density arrays allow optimized synthetic gradiometry as well as improved feature extraction and spatial filtering. Gradiometry may be used in combination with these ADFMR sensors to reduce or eliminate background noise in which the data from one or more secondary sensor (which may be further from the signal source) is subtracted from the primary sensor that is closer to the signal source. However, physical gradiometry, in which the outputs of the sensor are subtracted in analog, typically requires exact alignment of the primary and secondary sensors, and the noise floor is limited to the highest noise floor sensor of the pair. In contrast, synthetic gradiometry extends and improves upon physical gradiometry but cannot be easily employed in ambient conditions with most sensors due to their limited dynamic range.

Described herein two variations of synthetic gradiometey that may be used with any of these methods and apparatuses including ADFMR sensors. These techniques may be used together or separately. The first technique, described in greater detail below, uses local shielding specific to the ADFMR sensor to reduce or eliminate ambient fields. Thes second technique uses signal processing to isolate biosignals from the ADFMR sensor output and maximize signal to noise ratio.

In general, these methods and apparatuses may include the use of multiple sensors, including a primary ADFMR sensor and one or more secondary ADFMR sensors, similar to physical gradiometerty, but including further signal processing before combining/subtracting the signals. For example, a pair of sensors may be placed in proximity, much like in physical gradiometry, but the subtraction between the primary and secondary sensors may be applied after digitization. The weight of the signals between the primary and secondary sensors may be tuned in the digital domain prior to subtracting, including optimizing for signal to noise ratio (SNR). This tuning may be performed in an ongoing manner, in real time, and may include an analysis of the spectrum of the primary and/or secondary sensors, and performing linear subtraction. Thus tuning may maximize the decrease in ambient signal, and may minimize the ambient background. Any appropriate minimization technique may be used, including the use of an evolutionary algorithm, random walk, etc.

In some examples, the apparatus or method may include one or more sets of sensors that may be positioned to capture environmental noise in a region close to the subject along 3 orthogonal axes. This may allow characterization of the ongoing noise in real time, improving the discrimination of signal versus noise.

The tuning of the weights between the primary and secondary sensors in the digital domain may be performed in an ongoing manner, including recalibrating the relative weightings of the primary and secondary signals being subtracted with a predetermined frequency, or a variable (including adjustable) frequency. In some cases the frequency of calibration (“tuning”) may be adjusted based on environmental conditions, including movement/motion. Thus, in some case these apparatuses may include a motion sensor (e.g., an accelerometer or inertial measurement unit, IMU), which may be used to adjust the rate of tuning. For example, the methods and apparatuses may be configured to calibrate the weights of the primary and secondary sensors at a rate that is based on the motion of the sensor(s) (e.g., this rate may be 1 Hz for light movement, and may be performed as higher rates when more movement is detected (e.g., >10 Hz). Thus, these methods and apparatuses may help correct for noise (artifact) due to or correlated with movement. The rate may be adjusted as part of the signal processing used for the direct gradiometery.

As mentioned, the signal (output) of the one or more secondary sensors that is/are subtracted from the primary sensor(s) may be weighted or scaled, in the digital domain, prior to subtracting. Thus, any of these methods and apparatuses may apply a weight to the signals from each secondary sensor that may be optimized to further lower noise. In some cases this weight may be determined using an optimization technique, e.g., optimization algorithm, such as a differential evolution algorithm, to minimize the spectral density outside a range of frequencies of interest. The weighting may account for differences in sensitivity between individual sensors as well as differences in noise amplitudes detected in each sensor due to the variable distances from the noise source. As mentioned, this tuning or calibration of the weights may be updated at a tuning frequency. In some cases the tuning frequency is least once every 100 ms (e.g., at 10 Hz or greater) to enable continuous movement of the sensors. The tuning frequency may be dynamic, as mentioned above.

Thus, correcting the output of an ADFMR sensor may generally include subtracting a weighted signal/output based on the output of one or more secondary ADFMR sensors from the primary ADFMR sensor signal. A maximization algorithm may be used to see the weighting values. All of these steps may be performed in the digital domain.

For example, any of these methods, or apparatuses configured to perform them, may comprise synthetic gradiometry including calibrating (e.g. weighting) the signal to be subtracted from one or more secondary sensors. The calibration weights may initially be set to 1 (e.g., equally weighted). In some cases the spectral densities of the secondary sensor(s) and the primary sensor(s) may be calculated. The level of noise present in the signals may be determined; in some examples the noise level may be determined by integrating over the frequencies outside of the range of interest (e.g., one or more regions of frequencies not of interest) and that may be common for noise sources. Alternatively or additionally, the SNR may be estimated by dividing the amplitude of a known signal frequency (or frequency band) by the integrated noise. Other metrics can also be utilized.

The calibration weights may be updated to minimize this estimated level of noise and/or maximize the SNR. For example the calibration weight (“weights”) may be adjusted/updated using either gradient-based or stochastic methods that minimize the calculated level of noise, maximize the SNR, or any other technique appropriate for the selected optimization metric. In some cases a gradient based method may be used to calculate the gradient (derivative) used, whereas a stochastic method may generate random variables to search a space.

This process may be repeated until the noise level is deemed low enough (e.g., it is less than and/or equal to a threshold noise level). Alternatively or additionally, this process may be repeated until the noise level does not change more than a predetermined value between iterations, and/or until a set number of iterations have been performed.

Once this optimized weight level has been estimated as described above, the calibration weight for the secondary sensor outputs (signals) may be applied, and the weighted output subtracted from the primary sensor output. The calibration weights may be recalculated as described above at the weighting frequency, e.g., after waiting for the period of time determined by the weighting frequency, such as 100 ms as mentioned above. In some cases recalibration (adjusting the calibration weight) may be performed more frequently, and/or continuously, or less frequently (e.g., 1 Hz), as mentioned.

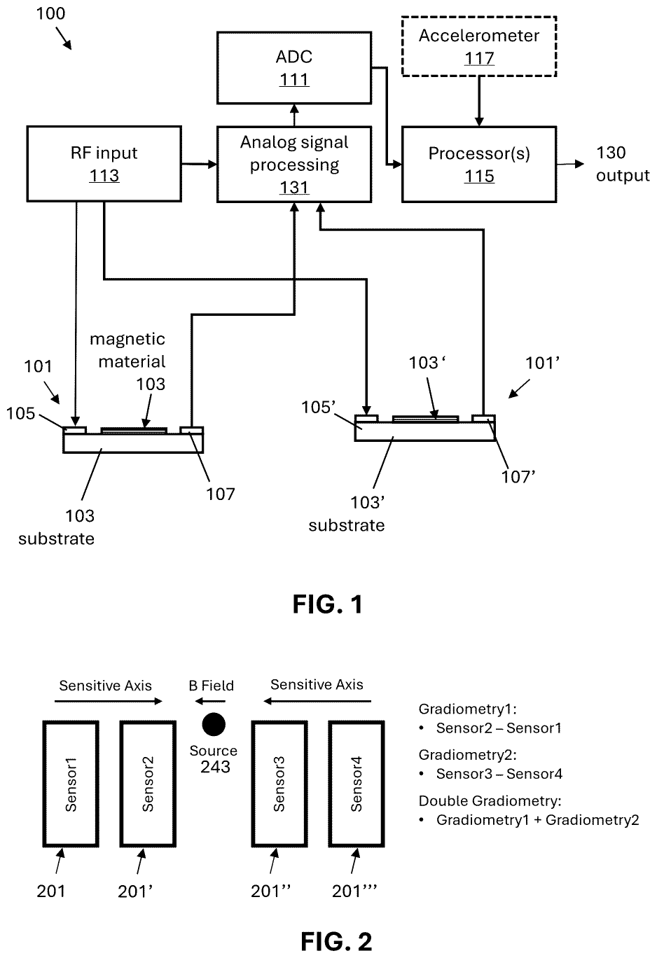

FIG. 1 schematically illustrates one example of an apparatus 100 including a primary and secondary ADFMR sensor set that are configured to sense relatively small magnetic fields such as those associated with biomagnetic signals, using synthetic gradiometry as described herein. In this example, two ADFMR sensors are shown for simplicity; more preferably additional (e.g., an array) of sensors may be used.

In general, an acoustically driven ferromagnetic resonance (ADFMR) sensor, also referred to equivalently herein as an ADFMR magnetometer, is a device that uses acoustic waves, such as surface acoustic waves (SAWs), to drive ferromagnetic resonance in order to exploit the coupling between strain and magnetization in a magnetostrictive material. The schematic ADFMR sensors 101, 101′ shown in FIG. 1 each include a magnetostrictive material 101, 101′ (e.g., magnet), a piezoelectric substrate 103, 103′. Any appropriate piezoelectric material may be used, including but not limited to LiNbO3. The apparatuses may each include least one input acoustic transducer 105, 105′ (e.g., an interdigitated transducer) on the piezoelectric substrate. The acoustic transducer is configured to activate the piezoelectric element to generate an acoustic wave. In any of these apparatuses the input acoustic transducer 105, 105′ may be interdigitated transducers with electrodes that are configured to excite surface acoustic waves (SAWs). The input acoustic transducer may be coupled (e.g., electrically coupled) to the source of input energy 113, such as a radio-frequency (RF) voltage source, to drive the application of acoustic energy through the device. The sensors may each also include at least one output acoustic transducer 107, 107′ on the piezoelectric substrate(s). The output acoustic transducer may be similar to the input acoustic transducer. The output acoustic transducer(s) 107, 107′ may electrically couple to processing circuitry, e.g., readout circuity, that is configured to detect a change in the acoustic wave to measure an unknown magnetic field to which the magnetostrictive element is exposed. In FIG. 1, the output of the transducers is passed on to the (optional) analog signal processor 131 that may process the output, which may include amplifying filtering, combining, etc. The apparatus also includes an analog-to-digital converter (ADC) 111 to convert the sensed signals (passed on from the analog signal processor 131) into the digital domain. One or more processor 115 may receive the digital signals and may weight the signals from the secondary (101′) sensors as described above, and subtract the weighted secondary signal from the primary (101) signal to produce the output 130, which may be stored, further processed and/or displayed. The apparatus may optionally include a motion sensor 117 (e.g., an accelerometer and/or IMU), as mentioned above, to adjust the weighting (calibration) frequency, as mentioned above. The methods and apparatuses described herein may modify an ADFMR sensor to enhance the sensitivity of the sensor by including additional components (including circuitry) not shown in the simplified example of FIG. 1.

The methods and apparatuses described herein are particularly useful in allowing a flexible form factor that may be adjustably or movably applied to a body (e.g., worn). Any of the apparatuses described herein may be configured as part of a wearable garment or accessory, and may permit adjustable/flexible positioning of the various sensors, including relative changes in the positions between primary and secondary ADFMR sensors. This is particularly surprising in comparison to other magnetic sensing systems or other sensing systems in general that reduce noise by attempting to subtract from a secondary sensor, which is widely thought that the primary and secondary sensors must be in fixed positions relative to each other. Thus, in general, the individual sensors (e.g., primary and secondary sensors) may be movably or flexibly connected to each other and to the other components of the apparatus (e.g., RF input, circuitry, etc.). The use of tuning, including real-time tuning and/or adaptive tuning, as described herein may permit this flexibility without significant loss of sensitivity and accuracy.

Although the example shown in FIG. 1 includes a single secondary sensor 101′, in some cases three or more (e.g., four) secondary sensors may be used, arranged in the different directions/axes. In any of these apparatuses and methods, synthetic gradiometry does not have to pre-define ‘signal’ and ‘reference’ sensors. For example any of these apparatuses may include an array of N sensors, and may perform subtraction operations among them. The apparatus or method may update those relationships dynamically. For example, for 4 sensors (e.g., indicated by A, B, C, D), the method or apparatus may dynamically have an output of E=A-B, F=C-D, or E=A-C, F=B-D, or E=A-B, F=C-E, G=D-E, etc. depending on dynamic positioning, noise, motion, etc.

Multiple sets of sensors may be used for gradiometer according to the techniques described herein. FIG. 2 schematically illustrates an example including four sensors 201, 201′, 201″, 201″′ that may be used as pairs for sensing the magnetic field from a source 243 emitting a magnetic field (B field). In this example, a single gradiometery configuration is shown by the configuration in which the output of sensor 2 is subtracted from the scaled/weighted output of sensor 1 (“Gradiometry1”). Similarly a second single gradiometery configuration is also shown, in which the output of sensor 3 is subtracted from the scaled/weighted output of sensor 4 (“Gradiometry2”). This example also illustrates a “double gradiometry”) configuration, in which the resulting outputs are combined, e.g., Gradiometry1+Gradiometry2.

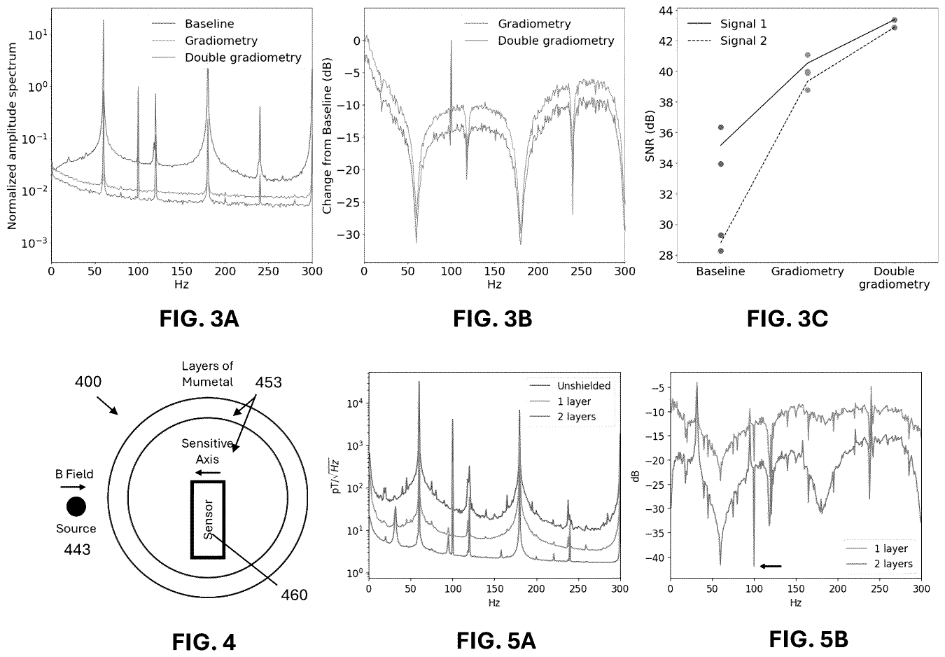

FIG. 3 illustrates and example of the reduction in ambient noise from the raw baseline recordings (FIGS. 3A and 3B) and the signal to noise ratio (SNR) gain (FIG. 3C) for an arrangement such as that shown in FIG. 2. In this example, gradiometry can reduce noise by up to 30 dB and provide an SNR gain of as much as 15 dB from baseline recordings, and typically provides more gain with smaller signals. Additional gradiometer setups, such as multidimensional gradiometry or various synthetic gradiometry methods, can be used in addition to the traditional gradiometry shown here. In general, the gradiometer described herein, which re-calibrates at some calibration frequency may provide highly robust results even at magnetic fields magnitudes that are within the ambient noise levels.

The methods and apparatuses described herein may also use extraction and/or filtering as part of the processes described herein, including feature extraction and spatial filtering of the output and/or the tuning. Gradiometry may have a difficult time properly removing noise from sources close to the signal source (<10 cm). Feature extraction and spatial filtering are digital signal processing techniques that can isolate the signals of interest by separating the sources. Feature extraction may perform blind source separation using premade assumptions about the signals, whereas spatial filtering may take the sensor position into account to recreate where and how the sources should be positioned to result in the composite signal recorded from each sensor. These techniques may allow both isolation and location of the signal sources, but prior to this work have not been employed for the purposes of magnetocardiography (MCG) or magnetomyography (MMG), in large part due to the lack of sensor density. The small size of the ADFMR magnetometer sensing elements described herein, e.g., approximately 0.1×1 mm sensitive area (e.g., 0.1×0.5 mm, 0.05×0.5 mm, 0.1 mm×0.1 mm, etc.), 3×3 mm packaged size (e.g., 4×4 mm, 5×5 mm, etc.) target will allow for high density resulting in more effective feature extraction and spatial filtering.

### Shielding

Also described herein are ADFMR sensors that include shielding, and in particular, small, light, and mobile shielding that is configured to attenuate environmental noise. The shielding may be configured to be removed, and may be configured to allow the sensors to be worn. In particular, the shielding described herein is configured to only partially enclose the ADFMR sensor, while common wisdom holds that shielding needs to completely enclose in sensor. However, the ADFMR sensors described herein may only partially enclose the sensor yet still achieve sufficient shielding.

In some cases the shielding may comprise one or more (e.g., two, three, four, etc.) metallic layers (e.g., foil layers). This metallic foil may be relatively thin, e.g., 0.1 mm thick or less (e.g., 0.3 mm or less, 0.2 mm or less, 0.08 mm or less, 0.07 mm or less, 0.06 mm or less, 0.05 mm or less, 0.04 mm or less, 0.03 mm or less, 0.02 mm or less, etc.). In some cases, a single layer or a couple (e.g., two) layers thin foil may be used. In some cases the foil shield layers does not completely enclose the sensor. For example a single layer of 0.1 mm thick continuous foil material may be used, resulting in a lightweight and flexible shield (e.g., foil-thin shielding).

To block magnetic fields, the sensor may be shielded in both directions of the recording axis. In some examples, the shielding may be attached to the body, and/or it can be attached to the sensing system. For example in some applications (e.g., a Magnetocardiography, MCG, application), the sensor array could be placed within a cylinder (e.g., that is open on both ends, or even just open on one end) of mumetal/shielding material to ensure that only magnetic fields originating near the opening of the shield are measured. In some cases the biomagnetic applications are focused on the brain, which is difficult to fully shield as the sensors are typically placed along the curvature of the scalp requiring a sphere around the entire head to properly block out environmental noise. As a result, most shielding applications have focused on magnetically shielded rooms to encase the entire subject within a magnetically isolated space. In any of the variations described herein, the sensor may be shielded by wrapping a shielding material that is relatively thin and flexible around the sensor and the region of the body to which the sensor is attached, to achieve MCG and MMG sensing using one or more (e.g., an array) of ADFMR sensors. For example, the sensor(s) and body region may be wrapped in a magnetic shielding material around the torso or the limb with the sensors within the shielded region to eliminate ambient noise without the need for a magnetically shielded room. In addition, shielding can be performed with a wearable form factor material due to the small size of the ADFMR magnetometers. Any appropriate magnetic shielding material may be used, including mu metals (e.g., alloys of nickel and iron, in some cases with small amounts of other elements like copper and molybdenum).

FIG. 4 schematically illustrates one example of a sensor 400 including a shield comprising one or more layers 453 of mu-metal forming a shield for an ADFMR sensor 460. In some examples even very thin layers of mu metals may provide efficacy of shielding these sensors. For example, a single layer of 0.1 mm thick mu-metal can eliminate broadband ambient noise by more than 10 dB, with 2 layers eliminating more than 20 dB. This was tested, using a configuration similar to that shown in FIG. 4, and the results are shown in FIGS. 5A-5B. For example, a noise source 443 was simulated to correspond to a nearby smartphone or computer, providing a 100 Hz tone at a distance of around 15 cm from the sensors. The 0.1 mm thick foil showed attenuation of more than 20 dB with one layer and more than 40 dB with 2 layers. Wearable form factors of shielded magnetic sensors have not been employed before, and may provide enhanced sensor function for biomagnetism in ambient conditions.

### Signal Processing

As discussed above, any of the methods and apparatuses described herein may be configured to include signal processing to isolate even low-intensity magnetic field signals (e.g., biosignals) and may maximize signal to noise ratio. In general, ambient magnetic field recordings have not been deeply explored due to the limitations of sensor technology. Thus, there is currently no signal processing pipeline for isolating biosignals and attenuating noise to maximize the signal to noise ratio. The methods and apparatuses described herein provide techniques and systems that may improve the signals of ADFMR sensors.

For example, any of these methods and apparatuses may use time domain filtering to remove line noise (e.g., 60 Hz) and its harmonics with an IIR notch filter and/or spectrum interpolation. As mentioned, this may be done in the digital domain. In some cases one or more analog filters may be used instead or additionally.

Alternatively or additionally, any of these methods and apparatuses may use a modified version of a two-step noise reduction (TSNR) technique which uses a predictive model, e.g., a model similar to a Wiener filter, to remove additive noise. Ambient noise data in the absence of signal may be used as baseline noise. For example, a TSNR technique may include a first step in which a noise reduction algorithm is applied. The noise reduction algorithm may be based on a decision-directed approach, which estimates the SNR and applies a spectral gain to reduce noise. The TSNR technique may then apply a second step, such as a refinement step that may further improve the SNR estimation by addressing potential biases from the first step, leading to better noise reduction results.

Alternatively or additionally, any of these methods and apparatuses may use an independent component analysis (ICA), principal component analysis (PCA), or other source separation technique (and/or a combination or sequence of multiple techniques) to separate signals from noise. A method accounting for sparsity, such as sparse dictionary learning, may be used as a higher density of sensors and a very small, localized signal means only a select number of sensors can detect the signal at all.

Alternatively or additionally, any of these methods and apparatuses may use a spatial filtering technique, such as linearly constrained maximum variance beamforming, using known positions and sensitive axes of each sensor and a model of the biological source (e.g., muscle fibers or brain structure) to determine which components are biosignals and which are noise.

In some cases all or just a sub-set of these techniques are used.

Alternatively or additionally, any of these methods and apparatuses may use one or more machine learning (e.g., deep learning) approaches to further denoise the extracted biosignals, such as a deep denoising autoencoder (DAE). A DAE may be trained on baseline signals recorded from within a magnetic shielded room as well as baseline signals recorded in ambient conditions. The model may assume that the ambient noise is injected noise and the recorded signals inside the shielded room are the ground truth signals, thus learning how to remove the various noise sources to match the signals recorded from the shielded room.

In some cases, any of these methods and apparatuses may use a generative adversarial network (GAN) that is trained such that one network attempts to denoise ambient recorded data and the other network attempts to classify between ambient and shielded room data. As both networks are trained the former network becomes better at removing ambient noise to match the shielded room data as it competes with the latter network, which we can then use for denoising biomagnetic data recorded in ambient conditions.

The application of such machine learning networks has not previously been used. The final trained network may provide a novel resource to denoise ambient magnetic noise for recording biomagnetic signals.

As mentioned above in the context of synthetic gradiometer, any of these methods and apparatuses may include one or more motion sensors (e.g., inertial measurement units and/or accelerometers) to track the movement and rotation of the sensors to inform the noise rejection techniques for dynamic situations.

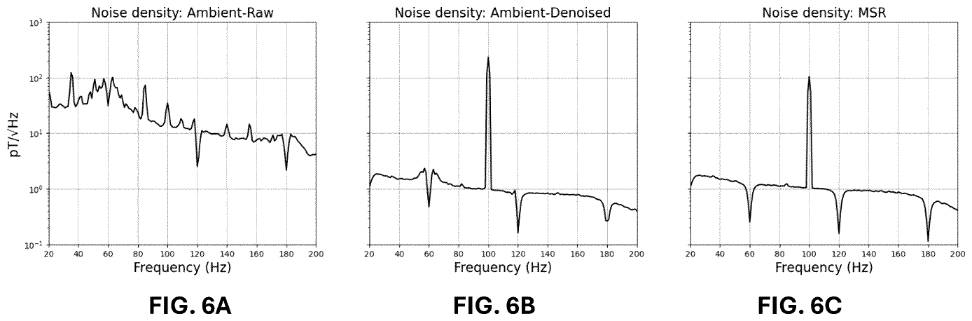

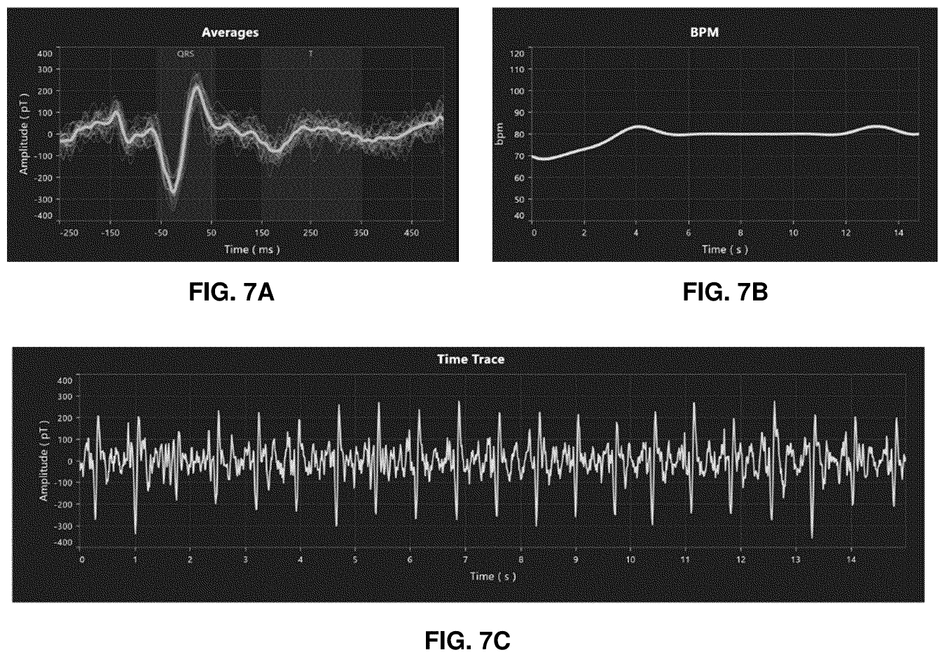

FIGS. 6A-6C illustrate examples of the use of denoising using the techniques described above (e.g., time-domain filtering using an IIR notch filter, TSNR, PCA and/or spatial filtering), showing a significant noise rejection (FIG. 6B, obtained using time domain filtering and TSNR). FIG. 6A shows the level of ambient/raw noise density, and FIG. 6C shows the noise density in a magnetically shielded room. The noise levels after applying the algorithm to data recorded in ambient conditions rival the noise levels recorded in a magnetically shielded room (i.e. sensor noise floor). In this example, a 10 mA, 100 Hz sinusoidal source current on a custom dipole was recorded with magnetic sensors inside a magnetic shielded room (MSR), shown in FIG. 6C, as well as in controlled (e.g., away from large magnetic fields) ambient conditions (FIG. 6A). After denoising (FIG. 6B) the 100 Hz peak was clearly visible in the ambient signal noise density similar to the noise density of the shielded condition. The difference in the peak signal is due to slightly different sensor positions relative to the dipole.

The methods and apparatuses described herein (e.g., the use of synthetic gradiometer, shielding with a flexible or thin-film/foil material, and/or denoising) may be used in combination or individually to improve the sensitivity of one or more magnetometers, and in particular ADFMR sensors. The combination of some or all of these apparatuses and techniques with one or more ADFMR sensors may enable recording of biomagnetic signals in ambient and ambulatory conditions. Examples of such biomagnetic signals may include (but are not limited to) heart signals (MCG), which has been an area of study with various types of magnetic sensors but has yet to be developed into a commercially viable technology, and magnetic muscle signals (magnetomyography, MMG), which has yet to be measured and characterized in unshielded conditions. The methods and apparatuses described herein may be used to successfully and robustly achieve both MCG and MMG using the techniques discussed herein in ambient conditions.

For example, FIGS. 7A-7C show examples of recordings of cardiac signals magnetocardiography (MCG) taken using an apparatus as described herein. In this example, an array of 14 third-party sensors that were aligned to record perpendicular to the ECG voltage (i.e. oriented from left shoulder to right hip) and synthetic gradiometry, time domain filtering, and ICA (a form of source separation) were used to obtain the data shown. FIG. 7A shows a series of averaged traces over time, and FIG. 7B shows beats per minute calculated using these methods. FIG. 7C shows an extracted real-time signal. The resulting signals compare very well with sensing using other modalities (e.g., ECG) and with measurements done in a fully shielded room.

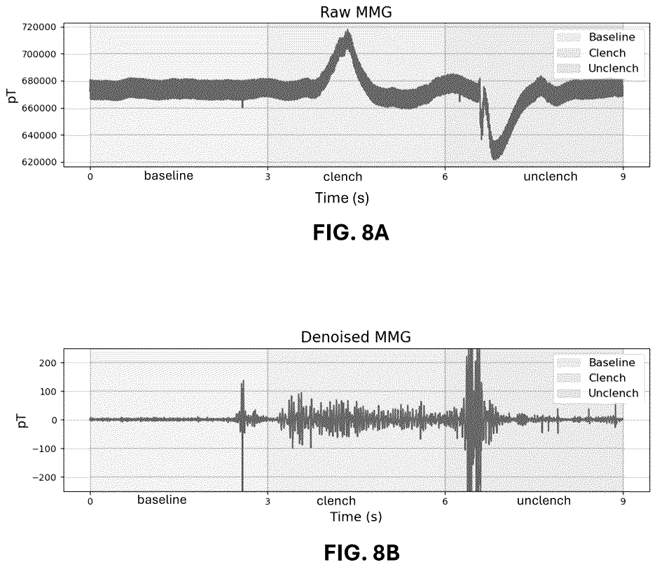

Similarly, FIGS. 8A-8B illustrate an example of measured muscle activity (MMG) recorded using an apparatus as described herein. In this example, a magnetometer was used with synthetic gradiometry, time domain filtering, TSNR, and independent component analysis (ICA) on that trace to obtain the data shown. In FIG. 8A the ambient MMG graph shows raw MMG, while FIG. 8B shows denoised MMG. The time traces correspond to MMG during baseline (3 seconds), clenching (3 seconds), and unclenching (3 seconds). There is a significant difference in signal amplitude between the raw signal (e.g., ranging up to micro-tesla due to ambient noise sources) and the denoised signal (in pT after removal of ambient noise). During unclenching, the high amplitude burst may be due to the sudden movement of the sensors from unclenching.

The methods and apparatuses described herein provide techniques that may be combined with one or more (e.g., an array) of ADFMR magnetometers, and in particular smaller form-factor (e.g., 5×5 mm or smaller) ADFMR magnetometers. The majority of techniques discussed above have not been previously used for ambient noise rejection while recording biomagnetic signals, and may provide surprisingly better results, particularly in the context of these small ADFMR sensors. For example, synthetic gradiometery has not been used in ambient conditions, possibly due to the dynamic range limitations of currently available magnetometers. In addition, thin, lightweight (and flexible, e.g., foil) shielding has not been used, particularly “incomplete” shielding that does not cover the full sensor. Instead, the art has focused on shielding full rooms, e.g., having walls that provide shielding. Shielding in a wearable form factor has never been employed, particularly not in the context of the compact ADFMR sensors described herein.

In general, feature extraction and spatial filtering accuracy and effectiveness may directly scale with the density of sensors as multiple sensors recording the same signal improves its extraction. Simulations have confirmed this assumption and have posited it as a large limitation of magnetic sensing, but it has yet to be demonstrated experimentally due to the large size of existing sensors.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of sensing a biomagnetic field under ambient conditions in an ambulatory subject, the method comprising:
receiving a primary signal from one or more primary acoustically driven ferromagnetic resonance (ADFMR) sensors worn on the subject;
receiving a secondary signal from one or more secondary ADFMR sensors worn on the subject;
weighting the secondary signal by a weighting factor;
periodically adjusting, at a tuning frequency, the weighting factor;
generating a corrected biomagnetic sensor signal by subtracting the primary signal from the weighted secondary signal; and
outputting the corrected biomagnetic sensor signal.

2. The method of claim 1, further comprising estimating a level of noise from a spectral density of the primary and/or secondary signal and weighting the secondary signal to minimize the estimated noise.

3. The method of claim 2, wherein weighting the secondary signal to minimize the estimated noise comprises using a gradient-based and/or stochastic technique to minimize the estimate noise.

4. The method of claim 1, wherein receiving the secondary signal comprises receiving the secondary signal from the one or more secondary ADFMR sensor that is flexibly connected to the one or more primary ADFMR sensors.

5. The method of claim 1, wherein outputting comprises continuously outputting.

6. The method of claim 1, wherein outputting comprises outputting in real time.

7. The method of claim 1, wherein the one or more secondary ADFMR sensors comprises three or more secondary ADFMR sensors that are arranged orthogonal to each other.

8. The method of claim 1, wherein the tuning frequency is adjusted based on input from a motion sensor.

9. The method of claim 1, wherein the tuning frequency is between 0.1 Hz and 10 Hz.

10. The method of claim 1, further comprising converting the primary signal and the secondary signal into the digital domain before generating the corrected biomagnetic signal.

11. The method of claim 1, further comprising shielding the one or more primary and one or more secondary ADFMR sensors.

12. The method of claim 11, wherein shielding comprises shielding under a mumetal foil.

13. The method of claim 1, further comprising denoising the corrected biomagnetic sensor.

14. A method of sensing a biomagnetic field under ambient conditions in an ambulatory subject, the method comprising:
receiving a primary signal from one or more primary acoustically driven ferromagnetic resonance (ADFMR) sensors worn on the subject;
receiving a secondary signal from one or more secondary ADFMR sensors worn on the subject;
estimating a level of noise from a spectral density of the primary and/or secondary signal;
weighting the secondary signal by a weighting factor to minimize the estimated noise using a gradient-based and/or stochastic technique;
periodically adjusting, at a tuning frequency, the weighting factor;
generating a corrected biomagnetic sensor signal by subtracting the primary signal from the weighted secondary signal; and
outputting the corrected biomagnetic sensor signal.

15. A method of sensing a biomagnetic field under ambient conditions in an ambulatory subject, the method comprising:
placing an acoustically driven ferromagnetic resonance (ADFMR) sensor on a region of a subject's body;
shielding, using a flexible foil shield, the ADFMR sensor by at least partially covering the ADFMR sensor with the flexible foil shield;
receiving a signal from the shielded ADFMR sensor worn on the subject;
generating a biomagnetic sensor signal from the signal; and
outputting the biomagnetic sensor signal.

16. The method of claim 15, further shielding comprises partially shielding the ADFMR sensor.

17. The method of claim 15, wherein the flexible foil shield comprises a mumetal foil.

18. The method of claim 15, wherein the flexible foil shield is between 0.01 and 0.2 mm thick.

19. The method of claim 15, wherein the flexible foil shield is approximately 0.1 mm thick.

20. The method of claim 15, wherein the flexible foil shield comprises between 1-4 layers of foil.
